(19) United States
(12) Patent Application Publication
(10) Pub. No.: US 2022/0370257 A1
(43) Pub. Date: Nov. 24, 2022

(54) ANGLED SAMPLING SWABS

(71) Applicant: Illinois Tool Works Inc., Glenview, IL (US)

(72) Inventors: Paul M. Blair, Jonesboro, GA (US); Lauren Conti, Plano, TX (US)

(21) Appl. No.: 17/715,878

(22) Filed: Apr. 7, 2022

Related U.S. Application Data
(60) Provisional application No. 63/173,112, filed on Apr. 9, 2021.

(51) Int. Cl. A61F 13/38; A61B 10/00

(52) U.S. Cl. CPC A61F 13/38; A61B 10/0051

(57) ABSTRACT

An example angled sampling swab includes: a swab head comprising an absorbent material for specimen sample collection; and a handle. The handle includes: a first handle portion, wherein the swab head is attached to the second handle portion; and a second handle portion comprising a notch enabling separation of part of the second handle portion to enable insertion of the swab head and the first handle portion into a transport vial, the second handle portion arranged at an angle of 80 degrees or greater and less than 170 degrees with respect to the first handle portion, such that the swab head is arranged at the same angle with respect to the first handle portion

### FIELD OF THE DISCLOSURE

The present application claims the benefit of U.S. Provisional Patent Application Ser. No. 63/173,112, filed Apr. 9, 2021, entitled “ANGLED SAMPLING SWABS.” The entirety of U.S. Provisional Patent Application Ser. No. 63/173,112 is expressly incorporated herein by reference.

### FIELD OF THE DISCLOSURE

This disclosure relates generally to sampling swabs and, more particularly, to angled sampling swabs.

### BACKGROUND

Sampling swabs are often used in the medical field to collect specimens from patients. Conventional sampling swabs are typically straight, such as for insertion into the nasal cavity, or deeper into the nasopharyngeal cavity.

### SUMMARY

Angled sampling swabs are disclosed, substantially as illustrated by and described in connection with at least one of the figures, as set forth more completely in the claims.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIGS. 1A-1D illustrate example angled flocked sampling swabs and angled foam sampling swabs, in accordance with aspects of this disclosure.

FIG. 2 illustrates an example sample collection from a neonatal patient fitted with an endotracheal tube holder using one of the example angled sampling swabs of FIGS. 1A-1D.

The figures are not necessarily to scale. Wherever appropriate, similar or identical reference numerals are used to refer to similar or identical components.

### DETAILED DESCRIPTION

Sampling swabs are used to collect sample secretions from the anterior nares and/or the mid turbinate regions of the nasal cavity. Conventional sampling swabs have straight handles. However, in certain situations, insertion of a sampling swab into the nasal cavity may be obstructed. For instance, babies in incubators of neonatal intensive care units (NICUs) may be provided with endotracheal tube holders, which extend across the face of the baby. Such endotracheal tube holders present an obstacle to proper insertion of a conventional sampling swab for nasal sample collection.

Disclosed angled sampling swabs may be advantageously used for collecting nasal secretions from babies in incubators in Neonatal Intensive Care Units (NICU) and/or in other instances in which obstructions to sample collection are present. Unlike conventional sampling swabs, which are straight, disclosed example angled sampling swabs have heads that are arranged at an angle between 80 degrees and 170 degrees, constructed of sterile flocked or foam material. Example angles between the swab heads and handles are 90 degrees, 120 degrees, and 135 degrees.

Disclosed angled sampling swabs may be used to collect sample secretions from the anterior nares and/or the mid turbinate portions of the nasal cavity. Some disclosed angled sampling swabs are not intended for nasopharyngeal sample collection.

Disclosed angled sampling swabs may further include a notch and/or other features on the handle to facilitate separation of the swab head from at least part of the handle, to permit the swab head to be inserted into a transport vial. In some examples, the handle is provided with a notch to provide a specific and consistent location on the handle for separation. The easy separation allows for transportation of the sample in an effective vial (either dry or in a vial filled with transport medium) to a lab or analytical device.

Disclosed example angled sampling swabs include a swab head having an absorbent material for specimen sample collection, and a handle. The handle includes a first handle portion, to which the swab head is attached. The handle further includes a second handle portion having a notch, which enables separation of part of the second handle portion to enable insertion of the swab head and the first handle portion into a transport vial. The second handle portion is arranged at an angle of 80 degrees or greater and less than 170 degrees with respect to the first handle portion, such that the swab head is also arranged at the same angle with respect to the second handle portion.

In some example angled sampling swabs, the second handle portion is sufficiently rigid to maintain the angle during specimen collection. In some example angled sampling swabs, the outer diameter of the swab head is at least 1 millimeter and less than 4.5 millimeters. In some example angled sampling swabs, the angle is between 85 degrees and 95 degrees. In some other example angled sampling swabs, the angle is between 130 degrees and 140 degrees.

In some examples, the notch is between 40mm and 110 mm from the end of the second handle portion closest to the swab head. In some example angled sampling swabs, the angle between the first handle portion and the second handle portion is between 1 and 20 mm from an end of the swab head closest to the vertex of the angle.

In some examples, the handle is an injection molded plastic handle. In some example angled sampling swabs, the handle is an extruded plastic handle. In some example angled sampling swabs, the second handle portion has a rounded end, wherein the swab head surrounds the rounded end. In some examples, the swab head is flocked and comprises nylon, polyester, or bi-component microfiber. In some examples, the swab head includes medical-grade, open-cell polyurethane foam. In some example angled sampling swabs, the swab head and the first handle portion have respective lengths that are less than 15 mm.

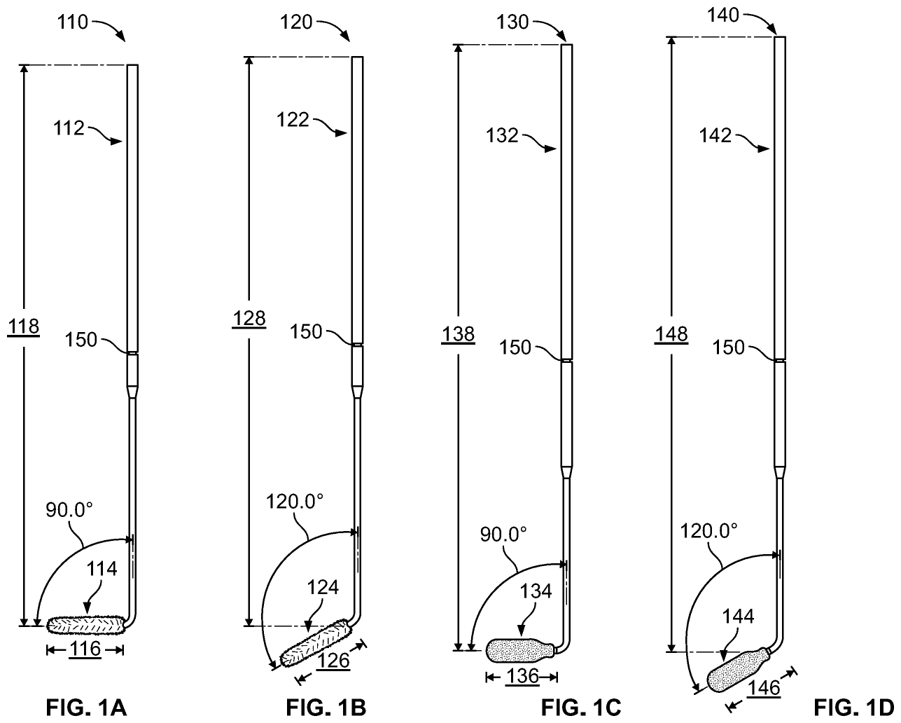

FIGS. 1A-1D illustrate example angled flocked sampling swabs 110 (FIG. 1A), 120 (FIG. 1B) and angled foam sampling swabs 130 (FIG. 1C), 140 (FIG. 1D). The example angled sampling swabs 110-140 are advantageous for obtaining sample specimens from a nasal cavity in the presence of an obstruction. A particularly advantageous use case involves obtaining nasal sample specimens from neonatal patients who are fitted with endotracheal tube holders.

Each of the example sampling swabs 110-140 includes a handle 112, 122, 132, 142 and a swab head 114, 124, 134, 144.

Each of the handles 112, 122, 132, 142 includes a first handle portion 116, 126, 136, 146, and a second handle portion 118, 128, 138, 148. The swab heads 114, 124, 134, 144 are attached, bonded, or otherwise coupled to the first handle portions 116, 126, 136, 146.

The swab heads 114, 124, 134, 144 are constructed using a soft, porous material for specimen sample collection. For example, the swab heads 114, 124 are a flocked nylon, polyester, bi-component microfiber, and/or any other suitable material. Conversely, the swab heads 134, 144 are an open-cell foam suitable for sample collection (e.g., about 100 pores per inch (PPI)).

The second handle portions 118, 128, 138, 148 are arranged at an angle of 80 degrees or greater, and less than 170 degrees, with respect to the first handle portion 116, 126, 136, 146. In the examples of FIGS. 1A-1D, the first handle portion 116 and the second handle portion 118 of the swab 110 are arranged at approximately a 90 degree angle. The first handle portion 126 and the second handle portion 128 of the swab 120 are arranged at approximately a 120 degree angle. The first handle portion 136 and the second handle portion 138 of the swab 130 are arranged at approximately a 90 degree angle. The first handle portion 146 and the second handle portion 148 of the swab 140 are arranged at approximately a 120 degree angle. Another example angle between the first and second handle portions (e.g., between the swab head and the second handle portion of the) is approximately 135 degrees.

Because the swab heads 114, 124, 134, 144 are attached to the first handle portions 116, 126, 136, 146, the swab heads 114, 124, 134, 144 are also arranged at the same respective angles with respect to the second handle portion 118, 128, 138, 148.

To facilitate sample collection and transportation, the second handle portions 118, 128, 138, 148 are capable of severing to reduce the length of the transport container (e.g., a vial) to fit the collected sample. In the examples of FIGS. 1A-1D, the handles 112, 122, 132, 142 are injection molded plastic handles. To aid in severing the second handle portions 118, 128, 138, 148, the second handle portions 118, 128, 138, 148 include respective notches 150. The notches 150 reduce the force required to sever the second handle portions 118, 128, 138, 148 and/or result in more consistent locations of severance. In some other examples, the handles 112, 122, 132, 142 are extruded plastic handles, which may require less force to sever than the injection molded plastic handles.

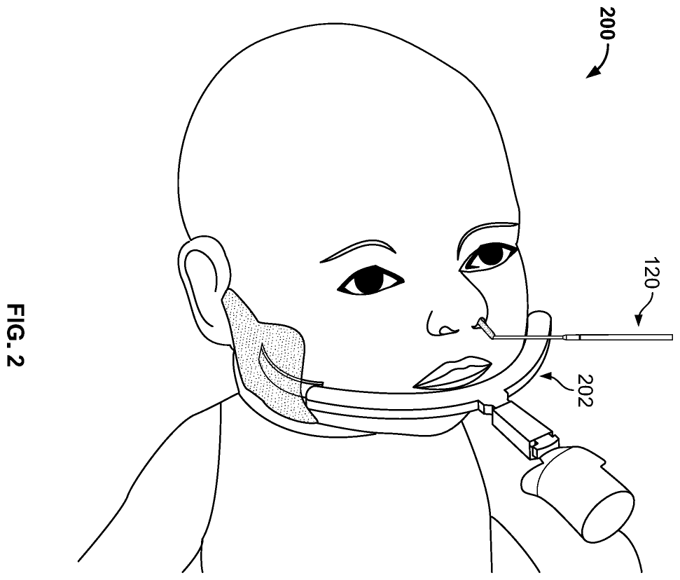

FIG. 2 illustrates an example sample collection from a neonatal patient 200 fitted with an endotracheal tube holder 202, using one of the example angled sampling swabs 120 of FIG. 1B. As illustrated in FIG. 2, the angled sampling swab 120 enables the swab head 124 to be placed inside of the nasal cavity of the patient with little or no interference from the endotracheal tube holder 202, and a significantly lower risk of unintentional striking or moving of the endotracheal tube holder 202 by the person manipulating the angled sampling swab 120.

To facilitate collection from neonatal patients, the outer diameter of the example swab heads is at least 1 millimeter and less than 4.5 millimeters, the vertex of the angle (i.e., (where the first handle portions 116, 126, 136, 146 and second handle portions 118, 128, 138, 148 meet) between the first handle portion and the second handle portion is less than 2 mm from an end of the swab head 114, 124, 134, 144 closest to the vertex of the angle.

While the present system has been described with reference to certain implementations, it will be understood by those skilled in the art that various changes may be made and equivalents may be substituted without departing from the scope of the present system. For example, block and/or components of disclosed examples may be combined, divided, re-arranged, and/or otherwise modified. In addition, many modifications may be made to adapt a particular situation or material to the teachings of the present disclosure without departing from its scope. Therefore, the present method and/or system are not limited to the particular implementations disclosed. Instead, the present method and/or system will include all implementations falling within the scope of the appended claims, both literally and under the doctrine of equivalents.

What is claimed is:

1. An angled sampling swab, comprising:
a swab head comprising an absorbent material for specimen sample collection; and
a handle, comprising:
a first handle portion, wherein the swab head is attached to the first handle portion; and
a second handle portion comprising a notch enabling separation of part of the second handle portion to enable insertion of the swab head and the first handle portion into a transport vial, the second handle portion arranged at an angle of 80 degrees or greater and less than 170 degrees with respect to the first handle portion, such that the swab head is arranged at the same angle with respect to the second handle portion.

2. The angled sampling swab as defined in claim 1, wherein the second handle portion is sufficiently rigid to maintain the angle during specimen collection.

3. The angled sampling swab as defined in claim 1, wherein the outer diameter of the swab head is at least 1 millimeter (mm) and less than 4.5 millimeters.

4. The angled sampling swab as defined in claim 1, wherein the angle is between 85 degrees and 95 degrees.

5. The angled sampling swab as defined in claim 1, wherein the angle is between 130 degrees and 140 degrees.

6. The angled sampling swab as defined in claim 1, wherein the notch is between 40 mm and 110 mm from the end of the second handle portion.

7. The angled sampling swab as defined in claim 1, wherein the angle between the first handle portion and the second handle portion is between 1 and 20 mm from an end of the swab head closest to the vertex of the angle.

8. The angled sampling swab as defined in claim 1, wherein the handle is an injection molded plastic handle.

9. The angled sampling swab as defined in claim 1, wherein the handle is an extruded plastic handle.

10. The angled sampling swab as defined in claim 1, wherein the second handle portion comprises a rounded end, wherein the swab head surrounds the rounded end.

11. The angled sampling swab as defined in claim 1, wherein the swab head is flocked and comprises nylon, polyester, or bi-component microfiber.

12. The angled sampling swab as defined in claim 1, wherein the swab head comprises medical-grade, open-cell polyurethane foam.

13. The angled sampling swab as defined in claim 1, wherein the swab head and the first handle portion have respective lengths that are less than 15 mm.
